SEQUENCE LISTING (1 sequence)

SEQ ID NO: 1 - protein, 204 aa, Homo sapiens
HTDLSGKVFVFPRESVTDHVNLITPLEKPLQNFTLCFRAYSDLSRAYSLFSYNTQGRDNELLVYKERVGEYSLYIGRHKVTSKVIEKFPAPVHICVSWESSSGIAEFWINGTPLVKKGLRQGYFVEAQPKIVLGQEQDSYGGKFDRSQSFVGEIGDLYMWDSVLPPENILSAYQGTPLPANILDWQALNYEIRGYVIIKPLVWV

(19) United States
(12) Patent Application Publication
(10) Pub. No.: US 2010/0266643 A1
(43) Pub. Date: Oct. 21, 2010

(54) PULMONARY AND NASAL DELIVERY OF SERUM AMYLOID P

(76) Inventors: W. Scott Willett, Doylestown, PA (US); Richard J. Caimi, Maple Glen, PA (US); Lynne Anne Murray, King of Prussia, PA (US)

Correspondence Address: ROPES & GRAY LLP, PATENT DOCKETING 39/41, ONE INTERNATIONAL PLACE, BOSTON, MA, 02110-2624, US

(21) Appl. No.: 12/752,627

(22) Filed: Apr. 1, 2010

Related U.S. Application Data
(60) Provisional application No. 61/211,609, filed on Apr. 1, 2009.

(51) Int. Cl. A61K 38/16; A61K 9/14; A61P 11/00

(52) U.S. Cl. 424/400; 514/1.1

(57) ABSTRACT

The disclosure relates to methods for delivery of serum amyloid P to the respiratory system. Pharmaceutical compositions comprising SAP suitable for respiratory delivery are also provided.

### CROSS-REFERENCE TO RELATED APPLICATIONS

This application claims the benefit of U.S. Provisional Application Ser. No. 61/211,609 filed Apr. 1, 2009. All the teachings of the above-referenced application is incorporated herein by reference.

### FIELD OF THE INVENTION

The disclosure relates to methods for delivery of serum amyloid P to the respiratory system. Pharmaceutical compositions comprising SAP suitable for respiratory delivery are also provided.

### BACKGROUND

Fibrosis is a condition characterized by the formation or development of excess fibrous connective tissue, excess extracellular matrix (ECM), excess scarring or excess collagen deposition in an organ or tissue as a reparative or reactive process. Pulmonary fibrosis describes a group of diseases whereby scarring occurs in the interstitium (or parenchymal) tissue of the lung. This tissue supports the air-sacs or alveoli, and during pulmonary fibrosis, these air sacs become replaced by fibrotic tissue, causing the tissue to become restructured and resulting in the reduced ability of the lung to transfer oxygen into the bloodstream. This relentless disease causes progressive structural remodeling of the lungs and is characterized clinically, for example, by increasing shortness of breath, chronic cough, progressive reduction in exercise tolerance and general fatigue. The disease can progress over a period of years, or progress very rapidly, resulting in patient debility, respiratory failure and eventually death. Development of fibrosis within the lungs can occur in patients afflicted with chronic inflammatory airway diseases, such as asthma, COPD (chronic obstructive pulmonary disease), emphysema, as well as in chronic smokers.

Chronic asthma is another fibrotic disorder characterized by structural changes within the lungs as a consequence of long-term, persistent asthma responses. The structural changes include airway smooth muscle hypertrophy and hyperplasia, collagen deposition to sub-epithelial basement membranes, hyperplasia of goblet cells, thickening of airway mucosa, and fibrosis. Tissue remodeling during chronic asthma results in airway obstruction that leads to progressive loss of lung function over time.

Current treatments available for treating fibrotic disorders include general immunosuppressive drugs, such as corticosteroids, and other anti-inflammatory treatments. However, the mechanisms involved in the regulation of fibrosis appear to be distinct from those of inflammation, and anti-inflammatory therapies are seldom effective in reducing or preventing fibrosis.

Recently, serum amyloid P (SAP) protein has been proposed as a therapeutic for treating disorders including fibrosis, see e.g., U.S. Patent Application No. 20070243163. SAP is a naturally occurring serum protein in mammals composed of five identical subunits or protomers that are non-covalently associated in a disc-like molecule. SAP binds to Fc receptors for IgG (FcγR), thereby providing an inhibitory signal for fibrocyte, fibrocyte precursor, myofibroblast precursor, and/or hematopoetic monocyte precursor differentiation.

A need thus remains for developing treatments to effectively target SAP to fibrotic tissue, such as in the lung.

### SUMMARY OF THE INVENTION

The present disclosure broadly relates to compositions, aerosolized compositions and methods for treating a variety of disorders affecting the respiratory tract. Both solid and liquid aerosolizable compositions of SAP are provided and are useful in treating SAP responsive disorders such as fibrosis and hypersensitivity disorders.

Pharmaceutical compositions of SAP are provided that are suitable for administration to the respiratory tract. Liquid compositions comprise from about 0.1 mg/ml to about 200 mg/ml of SAP, while solid compositions comprise from about 1% to about 100% w/w of SAP. In some embodiments, the compositions comprise from about 0.5 mg/ml to about 100 mg/ml, from about 1 mg/ml to about 50 mg/ml, or from about 1 to about 10 mg/ml. In some embodiments, the compositions comprise from about 10% to about 100%, from about 20% to about 90%, from about 30% to about 80%, or from about 40% to about 70% w/w of SAP.

In some embodiments, the compositions are suitable for administration to humans. In some embodiments, the composition is essentially pyrogen-free. In some embodiments, the composition comprises a pharmaceutically acceptable carrier. In some embodiments, the pharmaceutically acceptable carrier is sterile water.

In some embodiments, the composition comprises a lipid. In some embodiments, the composition comprises from about 0.1% to about 2% NaCl. In some embodiments, the composition comprises 1 mg/ml of SAP and 0.9% NaCl. In some embodiments, the composition comprises from about 1 mM to about 20 mM sodium phosphate. In some embodiments, the composition comprises from about 1 mM to about 20 mM sodium phosphate and from 1 to 10% sorbitol. In some embodiments, the composition comprises 1 mg/ml of SAP and 10 mM of sodium phosphate and 5% sorbitol. In some embodiments, the composition comprises 20 mg/ml of SAP and 10 mM of sodium phosphate and 5% sorbitol.

In some embodiments, the composition is dry powder suitable for delivery to the respiratory system comprising SAP and a pharmaceutically acceptable carrier.

In some embodiments, the composition comprises biodegradable microparticles comprising SAP and a pharmaceutically acceptable carrier.

In some embodiments, any of the above-described compositions are aerosolized. The aerosols are suitable for administration to the respiratory system. In some embodiments, the aerosol particles have a mass median aerodynamic diameter of less than about 10 microns. In some embodiments, the aerosol particles have a mass median diameter from about 1 to about 5 microns.

In some embodiments, the compositions are aerosolized with an appropriate inhalation device, such as a metered-dose inhaler; a dry powder inhaler, a nasal delivery device; or a nebulizer. In some embodiments, kits are provided comprising any of the above-described compositions and a suitable inhalation device. In some embodiments, inhalation devices are provided comprising any of the above-described compositions. In some embodiments, the inhalation device is selected a metered-dose inhaler; a dry powder inhaler, a nasal delivery device; or a jet, ultrasonic, pressurized or vibrating porous plate nebulizer.

Methods of administering SAP to a patient are provided, comprising aerosolizing a therapeutically effective amount of any of the SAP pharmaceutical compositions described herein. The methods are suitable for delivering SAP to the respiratory system of a patient. The methods are useful to treat any condition or disorder that benefits from SAP administration to the respiratory system.

In some embodiments, methods of treating respiratory fibrosis in a patient are provided, comprising administering to a patient in need thereof a therapeutically effective amount of any of the SAP pharmaceutical compositions described herein. In some embodiments, the respiratory fibrosis is selected from pulmonary fibrosis, idiopathic pulmonary fibrosis, chronic obstructive pulmonary disease, and chronic asthma.

In some embodiments, methods of treating a respiratory hypersensitivity disorder in a patient are provided, comprising administering to a patient in need thereof a therapeutically effective amount of any of the SAP pharmaceutical compositions described herein. In some embodiments, the respiratory hypersensitivity disorder is selected from allergic rhinitis, allergic sinusitis, and allergic asthma.

### BRIEF DESCRIPTION OF THE DRAWINGS

FIG. 1. Effects of intranasal SAP administration on bleomycin-induced lung fibrosis. Total lung collagen, measured as percent change in hydroxyproline, was used as a marker for fibrosis. Intranasal administration of SAP in mice elicits a significant decrease in lung fibrosis as compared to control treatment.

FIG. 2. SE-HPLC of nebulized SAP at 20 mg/mL in buffer.

FIG. 3. SE-HPLC of nebulized SAP at 1 mg/mL in buffer.

FIG. 4. SE-HPLC of nebulized SAP at 1 mg/mL in 0.9% NaCl.

FIG. 5. Exogenous SAP therapy prevented and reversed established airway hyperresponsiveness in a fungal asthma model. A. fumigatus-sensitized and conidia-challenged C57BL/6 mice received PBS, or hSAP via intraperitoneal (ip) injection every other day from days 0-15 (A) or 15-30 (B) after conidia, and airway resistance was measured following methacholine challenge using invasive airway resistance analysis (Buxco). Data are mean±SEM, n=5 mice/group. *P<0.05, ***P<0.001 compared with baseline airway resistance in the appropriate treatment group.

FIG. 6. Cytokine generation in splenocyte culture from cells isolated and simulated with aspergillus antigen and treated in vitro with hSAP. Spleen cells were isolated from animals 15 days (A) or 30 days (B) after intratracheal conidia challenge Animals were treated in vivo with hSAP (8 mg/kg, q2d, intranasal; filled bars) or PBS control (q2d, intranasal; open bars) for the last two weeks of the model. Cytokines were measured by ELISA using standard techniques.

FIG. 7. FoxP3 expression in pulmonary draining lymph nodes (A and B) or splenocyte cultures (C). A and B are from draining lymph nodes from the lung taken at day 15 from animals treated with PBS (control), or animals treated with SAP (+SAP) and stained for FoxP3. C is from splenocyte cultures that were stimulated with Aspergillus antigen in vitro in the presence or absence of SAP in vitro (0.1-10 μg/ml) for 24 hours. Total FoxP3 expression was quantitated using real time RT-PCR.

FIG. 8. Effects of SAP in vivo and in vitro on IL-10 and antigen recall. Mice were sensitized and challenged with Aspergillus fumigatus in vivo and treated with control (PBS, ip, 2qd, open bars) or SAP (5 mg/kg, ip q2d, filled bars) on days 15-30 post-live conidia challenge. At day 30 mice were killed, A. total lung IL-10 was measured by luminex, B-E. single cell splenocyte cultures were stimulated in vitro with Aspergillus fumigatus antigen, in the presence or absence on SAP and cell-free supernatants assessed for B. IL-10, C. IL-4, D. IL-5 and E. IFN-γ protein levels by specific ELISA. SAP treated animals (ip, 2qd on days 15-30) had enhanced levels of IL10 in the lungs in comparison to asthma control (PBS, ip, q2d, on days 15-30) and compared to native, non-allergic lung. Further there was a diminished antigen recall response, indicating enhanced T regulatory cell number and/or function.

### DETAILED DESCRIPTION OF THE INVENTION

### Overview

Aerosolized drug delivery provides certain advantages, such as safety and efficacy, compared to systemic drug delivery. For example, since the drug is delivered directly to the target region, the amount of aerosolized drug needed to assert its therapeutic effect is typically lower than the systemic dose because the systemic dose must account for delivery of the drug throughout the whole body rather than only to the organ where the treatment is needed. Additionally, since systemic delivery is avoided, there are none or fewer undesirable secondary effects. Finally, aerosolized drug delivery may increase patient compliance relative to intravenous systemic dosing.

Despite all these advantages, attempts to substitute systemic treatments with aerosolized drug delivery have met with only partial success because some drugs are not well tolerated by lungs and/or are not efficiently aerosolized.

The disclosure is based, in part, on the discovery that inhalation of serum amyloid P (SAP) protein is an effective method of delivery. The examples demonstrate that intranasal dosing of SAP was found to effectively treat lung fibrosis in an allergic airway disease model. The disclosure also demonstrates that the SAP protein can be aerosolized by conventional nebulizers.

The disclosure provides, inter alia, compositions and methods for the delivery of SAP to the respiratory system of a patient. Aerosolized administration of SAP delivers the protein directly to the target site, while minimizing systemic bioavailability. Nasal and pulmonary delivery systems are well known in the art and any suitable inhalation device may be used to administer SAP. Respiratory tract administration of SAP may be used to treat any condition or disorder that benefits from the biological effects of SAP.

### DEFINITIONS

The articles “a” and “an” are used herein to refer to one or to more than one (i.e., to at least one) of the grammatical object of the article.

The terms “comprise” and “comprising” are used in the inclusive, open sense, meaning that additional elements may be included.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably with, the phrase “such as but not limited to”.

“Mass median diameter” or “MMD” is a measure of the average size of a dispersed particle. MMD values can be determined by any conventional method such as laser diffractometry, electron microscopy, and centrifugal sedimentation.

“Mass median aerodynamic diameter” or “MMAD” is a measure of the aerodynamic size of a dispersed particle. The aerodynamic diameter is used to describe an aerosolized powder in terms of its settling behavior, and is the diameter of a unit density sphere having the same settling velocity, generally in air, as the particle. The aerodynamic diameter encompasses particle shape, density and physical size of a particle. As used herein, MMAD refers to the midpoint or median of the aerodynamic particle size distribution of an aerosolized powder determined by cascade impaction.

MMD and MMAD may differ from one another, e.g. a hollow sphere produced by spray drying may have a greater MMD than its MMAD.

A composition that is “suitable for pulmonary delivery” refers to a composition that is capable of being aerosolized and inhaled by a subject so that a portion of the aerosolized particles reaches the lungs to permit penetration into the alveoli. Such a composition is considered to be “respirable” or “inhalable”.

As used herein, “treating” refers to obtaining a desired pharmacologic and/or physiologic effect. The effect may be prophylactic in terms of completely or partially preventing a disorder or symptom thereof and/or may be therapeutic in terms of a partial or complete cure for a disorder and/or adverse affect attributable to the disorder. “Treating” includes: (a) increasing survival time; (b) decreasing the risk of death due to the disease; (c) preventing the disease from occurring in a subject which may be predisposed to the disease but has not yet been diagnosed as having it; (d) inhibiting the disease, i.e., arresting its development (e.g., reducing the rate of disease progression); and (e) relieving the disease, i.e., causing regression of the disease.

As used herein, a composition that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, the terms “subject” and “patient” are used interchangeable and refer to animals including mammals including humans. The term “mammal” includes primates, domesticated animals including dogs, cats, sheep, cattle, goats, pigs, mice, rats, rabbits, guinea pigs, horses, captive animals such as zoo animals, and wild animals.

### Pharmaceutical Compositions of Serum Amyloid P

In one aspect, the disclosure provides pharmaceutical compositions of SAP suitable for delivery to the respiratory tract. Naturally occurring SAP is a pentamer comprising five human SAP protomers. The sequence of the human SAP subunit is depicted in SEQ ID NO: 1 (amino acids 20-223 of Genbank Accession No. NP_{—}001630, signal sequence not depicted)

(SEQ ID NO: 1)

HTDLSGKVFVFPRESVTDHVNLITPLEKPLQNFTLCFRAYSDLSRAYSLF
SYNTQGRDNELLVYKERVGEYSLYIGRHKVTSKVIEKFPAPVHICVSWES
SSGIAEFWINGTPLVKKGLRQGYFVEAQPKIVLGQEQDSYGGKFDRSQSF
VGEIGDLYMWDSVLPPENILSAYQGTPLPANILDWQALNYEIRGYVIIKP
LVWV.

The term “SAP protomer” is intended to refer to a polypeptide that is at least 60%, at least 70%, at least 80%, at least 85%, at least 90%, at least 95%, at least 97%, at least 99% or 100% identical to human SAP protomer (SEQ ID NO:1), as determined using the FASTDB computer program based on the algorithm of Brutlag et al. (Comp. App. Biosci., 6:237-245 (1990)). In a specific embodiment, parameters employed to calculate percent identity and similarity of an amino acid alignment comprise: Matrix=PAM 150, k-tuple=2, Mismatch Penalty=1, Joining Penalty=20, Randomization Group Length=0, Cutoff Score=1, Gap Penalty=5 and Gap Size Penalty=0.05. The term “SAP protomer” encompasses functional fragments and fusion proteins comprising any of the preceding. Generally, an SAP protomer will be designed to be soluble in aqueous solutions at biologically relevant temperatures, pH levels and osmolarity. The protomers that non-covalently associate together to form SAP may have identical amino acid sequences and/or post-translational modifications or, alternatively, individual protomers may have different sequences and/or modifications.

In some embodiments, pharmaceutical compositions are provided comprising SAP, or a functional fragment thereof. In some embodiments, pharmaceutical compositions are provided comprising an SAP variant. In some aspects, the amino acid sequence of a SAP variant may differ from SEQ ID NO: 1 by one or more non-conservative substitutions. In other aspects, the amino acid sequence of a SAP variant may differ from SEQ ID NO: 1 by one or more conservative substitutions. As used herein, “conservative substitutions” are residues that are physically or functionally similar to the corresponding reference residues, i.e., a conservative substitution and its reference residue have similar size, shape, electric charge, chemical properties including the ability to form covalent or hydrogen bonds, or the like. Preferred conservative substitutions are those fulfilling the criteria defined for an accepted point mutation in Dayhoff et al., Atlas of Protein Sequence and Structure 5:345-352 (1978 & Supp.). Examples of conservative substitutions are substitutions within the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine. Additional guidance concerning which amino acid changes are likely to be phenotypically silent can be found in Bowie et al., Science 247:1306-1310 (1990).

Variants and fragments of SAP that retain biological function are useful in the pharmaceutical compositions and methods described herein. In some embodiments, a variant or fragment of SAP binds FcγRI, FcγRIIA, and/or FcγRIIIB. In some embodiments, a variant or fragment of SAP inhibits one or more of fibrocyte, fibrocyte precursor, myofibroblast precursor, and/or hematopoetic monocyte precursor differentiation.

In some embodiments, the pharmaceutical compositions comprise human SAP.

Pharmaceutical compositions suitable for respiratory delivery of SAP may be prepared in either solid or liquid form. Suitable pharmaceutical compositions comprising SAP are described in Publication No. 20070065368, which is hereby incorporated by reference. Exemplary compositions comprise SAP with one or more pharmaceutically acceptable carriers and, optionally, other therapeutic ingredients. The carrier(s) must be “pharmaceutically acceptable” in the sense of being compatible with the other ingredients of the composition and not eliciting an unacceptable deleterious effect in the subject. Such carriers are described herein or are otherwise well known to those skilled in the art of pharmacology. In some embodiments, the pharmaceutical compositions are pyrogen-free and are suitable for administration to a human patient. In some embodiments, the pharmaceutical compositions are irritant-free and are suitable for administration to a human patient. In some embodiments, the pharmaceutical compositions are allergen-free and are suitable for administration to a human patient. The compositions may be prepared by any of the methods well known in the art of pharmacy.

Liquid pharmaceutical compositions for producing an aerosol or spray may be prepared by combining SAP with a pharmaceutically acceptable carrier, such as sterile pyrogen-free water or allergen-free water. Liquid compositions typically have a pH that is compatible with physiological administration, such as pulmonary or nasal administration. In some embodiments, the liquid composition has a pH ranging from about 3 to about 7, or from about 4 to about 6. Liquid compositions also typically have an osmolality that is compatible with physiological administration, such as pulmonary or nasal administration. In some embodiments, the liquid composition has an osmolality ranging from about 90 mOsmol/kg to about 500 mOsmol/kg, 120 mOsmol/kg to about 500 mOsmol/kg, or from about 150 mOsmol/kg to about 300 mOsmol/kg.

In some embodiments, a liquid composition comprises from about 0.5 to about 100 mg/ml, from about 1 to about 50 mg/ml, or from about 10 to about 30 mg/ml of SAP. In some embodiments, a liquid composition comprises about 1, 5, 10, 20, 30, 40, or 50 mg/ml of SAP.

In some embodiments, a liquid composition comprising SAP further comprises from about 0.1% to about 5%, from about 0.1% to about 2%, or about 0.9% NaCl.

In some embodiments, a liquid composition comprising SAP further comprises from about 0.1 to about 50 mM, from about 1 to 20 mM, or about 10 mM sodium phosphate.

In some embodiments, the liquid composition comprising SAP further comprises 10 mM sodium phosphate, 5% sorbitol and has a pH of 7.5.

Suitable dry compositions of SAP are composed of aerosolizable particles effective to penetrate into the respiratory system of a patient. These dry powder pharmaceutical compositions comprise SAP in a dry form of appropriate particle size, or within an appropriate particle size range, for respiratory delivery. In some embodiments, the particles have a mass median aerodynamic diameter (MMAD) of less than about 100, 50, 10, 5, 4, 3.5, or 3 μm. The mass median aerodynamic diameters of the powders will characteristically range from about 0.1-10 μm, about 0.2-5.0 μm, about 1.0-4.0 μm, or from about 1.5 to 3.0 μm.

Dry powder devices typically require a powder mass in the range from about 1 mg to 20 mg to produce a single aerosolized dose (“puff”). If the required or desired dose of the biologically active agent is lower than this amount, the powdered active agent will typically be combined with a pharmaceutical dry bulking powder to provide the required total powder mass. Preferred dry bulking powders include sucrose, lactose, dextrose, mannitol, glycine, trehalose, human serum albumin (HSA), and starch. Other suitable dry bulking powders include cellobiose, dextrans, maltotriose, pectin, sodium citrate, and sodium ascorbate.

In some embodiments, the dry powders will have a moisture content below about 20% by weight, below about 10% by weight, or below about 5% by weight. Such low moisture-containing solids tend to exhibit greater stability upon packaging and storage.

In some embodiments, the dry powders comprise from about 10% to about 100% w/w of SAP. In some embodiments, the dry powders comprise from about 20% to about 90%, from about 30% to about 80%, or from about 40% to about 70% w/w of SAP.

Respirable powders can be produced by a variety of conventional techniques, such as jet milling, spray drying, solvent precipitation, supercritical fluid condensation, lyophilization, vacuum drying, air drying, or other forms of evaporative drying. Spray drying of the compositions is carried out, for example, as described generally in the “Spray Drying Handbook”, 5th ed., K. Masters, John Wiley & Sons, Inc., NY, N.Y. (1991), and in WO 97/41833 and WO 96/32149, the contents of which are incorporated herein by reference.

Once formed, the dry powder compositions may be maintained under dry (i.e., relatively low humidity) conditions during manufacture, processing, and storage. Irrespective of the drying process employed, the process will preferably result in inhalable, highly dispersible particles comprising SAP.

The pharmaceutical compositions, both solid and liquid, comprising SAP may further include flavoring agents, taste-masking agents, inorganic salts (for example sodium chloride), antimicrobial agents (for example benzalkonium chloride), sweeteners, antioxidants, antistatic agents, surfactants (for example polysorbates such as “TWEEN 20” and “TWEEN 80”), sorbitan esters, lipids (for example phospholipids such as lecithin and other phosphatidylcholines, phosphatidylethanolamines), fatty acids and fatty esters, steroids (for example cholesterol), and chelating agents (for example EDTA, zinc and other such suitable cations). Other pharmaceutical excipients and/or additives suitable for use in the compositions include polyvinylpyrrolidones, celluloses and derivatized celluloses such as hydroxymethylcellulose, hydroxyethylcellulose, and hydroxypropylmethylcellulose, Ficolls (a polymeric sugar), hydroxyethylstarch, dextrates (e.g., cyclodextrins, such as 2-hydroxypropyl-β-cyclodextrin and sulfobutylether-β-cyclodextrin), polyethylene glycols, and pectin. Additional excipients and/or additives may be found in “Remington: The Science & Practice of Pharmacy”, 19^{th} ed., Williams & Williams, (1995), and in the “Physician's Desk Reference”, 52^{nd} ed., Medical Economics, Montvale, N.J. (1998), both of which are incorporated herein by reference in their entireties.

To enhance delivery of SAP, compositions may also contain a hydrophilic low molecular weight compound as a base or excipient. Such hydrophilic low molecular weight compounds provide a passage medium through which SAP may diffuse through the base to the body surface where SAP is absorbed. The molecular weight of the hydrophilic low molecular weight compound is generally not more than 10,000 and preferably not more than 3,000 Da. Exemplary hydrophilic low molecular weight compound include polyol compounds, such as oligo-, di- and monosaccarides such as sucrose, mannitol, lactose, L-arabinose, D-erythrose, D-ribose, D-xylose, D-mannose, D-galactose, lactulose, cellobiose, gentibiose, glycerin and polyethylene glycol. Other examples of hydrophilic low molecular weight compounds useful as carriers include N-methylpyrrolidone, and alcohols (e.g., oligovinyl alcohol, ethanol, ethylene glycol, propylene glycol, etc.). These hydrophilic low molecular weight compounds can be used alone or in combination with one another or with other active or inactive components of the formulation.

In some embodiments, SAP is administered in a time release formulation, for example in a composition which includes a slow release polymer. SAP can be prepared with carriers that will protect against rapid release. Examples include a controlled release vehicle, such as a polymer, microencapsulated delivery system, or bioadhesive gel. Alternatively, prolonged delivery of SAP may be achieved by including in the composition agents that delay absorption, for example, aluminum monostearate hydrogels and gelatin.

In some embodiments, the pharmaceutical composition comprising SAP further comprises a lipid. SAP may be administered in the form of liposome delivery systems, such as small unilamellar vesicles, large unilamellar vesicles, and multilamellar vesicles.

The liposomes can be formed from synthetic, semi-synthetic, or naturally-occurring lipids, including phospholipids, tocopherols, sterols, fatty acids, glycolipids, anionic lipids, and cationic lipids. Exemplary lipids include phosphatidylcholine, phosphatidylglycerol, phosphatidylinositol, phosphatidylserine, phosphatidylethanolamine, and phosphatidic acid; sterically modified phosphatidylethanolamines, dimyristoylphosphatidycholine, dimyristoylphosphatidyl-glycerol, dipalmitoylphosphatidylcholine, dipalmitoylphosphatidylglycerol, distearoylphosphatidylcholine, distearoylphosphatidylglycerol, dioleylphosphatidyl-ethanolamine, palmitoylstearoylphosphatidylcholine, palmitoylstearoylphosphatidylglycerol, triacylglycerol, diacylglycerol, sphingosine, ceramide, sphingomyelin, and mono-oleoyl-phosphatidylethanolamine.

In some embodiments, a microparticulate system is used to deliver SAP to the respiratory system. The system comprises biodegradable microparticles comprising SAP and a pharmaceutically acceptable carrier. The term “microparticles” includes microspheres (uniform spheres), microcapsules (having a core and an outer layer of polymer), and particles of irregular shape. Microparticulate drug delivery systems are well-known in the art. In some embodiments, drug delivery is achieved by encapsulation of SAP in microparticles.

Any of a number of polymers can be used to form the microparticles. Polymers are preferably biodegradable within the time period over which release is desired or relatively soon thereafter, generally in the range of one year, more typically a few months, even more typically a few days to a few weeks. Biodegradation can refer to either a breakup of the microparticle, that is, dissociation of the polymers forming the microparticles and/or of the polymers themselves. In some embodiments, the polymers are selected from one or more of diketopiperazines; poly(hydroxy acids) such as poly(lactic acid), poly(glycolic acid) and copolymers thereof; polyanhydrides; polyesters such as polyorthoesters, polyamides; polycarbonates; polyalkylenes, such as polyethylene, polypropylene, poly(ethylene glycol), poly(ethylene oxide), and poly(ethylene terephthalate); poly vinyl compounds such as polyvinyl alcohols, polyvinyl ethers, polyvinyl esters, polyvinyl halides, polyvinylpyrrolidone, polyvinylacetate, and poly vinyl chloride; polystyrene; polysiloxanes; polymers of acrylic and methacrylic acids including poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyurethanes and co-polymers thereof; celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxyethyl cellulose, cellulose triacetate, and cellulose sulphate sodium salt; poly(valeric acid); and poly(lactide-co-caprolactone); natural polymers including alginate and other polysaccharides including dextran and cellulose; collagen; albumin and other hydrophilic proteins, zein and other prolamines and hydrophobic proteins; copolymers and mixtures thereof; bioadhesive polymers including bioerodible hydrogels; polyhyaluronic acids; casein; gelatin; glutin; polyanhydrides; polyacrylic acid; alginate; chitosan; and polyacrylates.

In one aspect, the disclosure provides aerosols comprising SAP. Aerosols are composed of liquid or solid particles that are suspended in a gas (typically air), typically as a result of actuation (or firing) of an inhalation device such as a dry powder inhaler, an atomizer, a metered-dose inhaler, or a nebulizer. Aerosols may be generated using any device suitable for producing respirable particles in order to aerosolize a pharmaceutical composition of SAP. Generally, aqueous formulations are aerosolized by spray pumps or nebulizers, propellant-based systems use suitable pressurized metered-dose inhalers, and dry powders may be dispersed with dry powder inhaler devices. In some embodiments, the aerosols comprise liquid particles of SAP. In some embodiments, the aerosols comprise dry particles of SAP. In some embodiments, the aerosols are generated by aerosolizing any pharmaceutical composition that is described herein.

In some embodiments, at least 10, 20, 30, or 40% by weight of the SAP in an SAP liquid pharmaceutical composition is aerosolized.

In some embodiments, at least 50, 60, 70, 80, 90, 95, 98, 99, or 100% of the SAP in the aerosol is in monomeric form, as determined, e.g., by SE-HPLC (see Example 2).

The optimum particle size of aerosolized SAP is dependent on the tissue to be targeted. Particles larger than 5 micron are deposited in upper airways, while particles smaller than around 1 micron are delivered into the alveoli and may get transferred into the systemic blood circulation. In some embodiments, aerosolized SAP particles have a mass median aerodynamic diameter of about 0.05 to about 100 micron, about 1 to about 20 micron, or less than about 10 micron. In some embodiments, aerosolized SAP particles have a mass median diameter from about 0.05 to about 100 micron, about 1 to about 20 micron, or about 1 to about 5 micron. Small aerodynamic diameters are generally achieved by a combination of optimized drying conditions and choice and concentration of excipients, parameters which are well-known to one skilled in the art.

In some embodiments, the composition to be aerosolized is mixed with a propellant, such as fluorotrichloromethane, dichlorodifluoromethane, dichlorotetrafluoroethane, or a hydrofluoroalkane, such as hydrofluoroalkane 134a (HFA 134a, 1,1,1,2-tetrafluoroethane) and hydrofluoroalkane 227 (HFA 227, 1,1,1,2,3,3,3-heptafluoropropane).

### Inhalation Devices for Delivery of Serum Amyloid P

As those skilled in the art will appreciate, many conventional methods and apparatuses are available for administering pharmaceutical compositions of SAP to the respiratory system of a patient. Inhalation devices suitable to deliver SAP to the respiratory system of a patient include, e.g., nebulizers, dry powder inhalers, and nasal sprays.

Aerosols of liquid particles comprising SAP may be produced by any suitable means, such as with a nebulizer. See, e.g. U.S. Pat. No. 4,501,729. Nebulizers transform solutions or suspensions into a therapeutic aerosol mist either by means, e.g., of acceleration of a compressed gas, typically air or oxygen, through a narrow venturi orifice or by means of ultrasonic agitation.

Typical devices include jet nebulizers, ultrasonic nebulizers, pressurized aerosol generating nebulizers, and vibrating porous plate nebulizers. A jet nebulizer utilizes air pressure to break a liquid solution into aerosol droplets. An ultrasonic nebulizer works by a piezoelectric crystal that shears a liquid into small aerosol droplets. Pressurized systems general force solutions through small pores to generate small particles. A vibrating porous plate device utilizes rapid vibration to shear a stream of liquid into appropriate droplet sizes. A variety of commercially available devices are available. Representative suitable nebulizers include the eFlow™ nebulizer available from Pari Inovative Manufactures, Midlothian, Va.; the iNeb™ nebulizer available from Profile Drug Delivery of West Sussex, United Kingdom; the Omeron MicroAir™ nebulizer available from Omeron, Inc. of Chicago, Ill. and the AeroNebGo™ nebulizer available from Aerogen Inc. of Mountain View, Calif.

Patients maintained on a ventilating apparatus can be administered an aerosol of respirable particles by nebulizing the liquid composition and introducing the aerosol into the inspiratory gas stream of the ventilating apparatus, as described in U.S. Pat. No. 4,832,012.

In some embodiments, the liquid pharmaceutical composition is delivered to a patient's respiratory system as a nasal spray. Exemplary devices are disclosed in U.S. Pat. No. 4,511,069, hereby incorporated by reference. The compositions may be presented in multi-dose containers, for example in the sealed dispensing system disclosed in U.S. Pat. No. 4,511,069. Other suitable nasal spray delivery systems have been described in Transdermal Systemic Medication, Y. W. Chien, Elsevier Publishers, New York, 1985; and in U.S. Pat. No. 4,778,810.

In some embodiments, the pharmaceutical composition is a dry powder and any solid particulate medicament aerosol generator may be used to deliver the composition to the respiratory system of a patient. Dry powders can be administered to a patient via conventional dry powder inhalers (DPI) which rely on the patient's breath, i.e., upon pulmonary or nasal inhalation, to disperse the powder into an aerosolized amount. Dry powder inhalation devices include those described in European Patent Nos. EP129985, EP472598, and EP 467172 and U.S. Pat. Nos. 5,522,385, 5,458,135, 5,740,794, and 5,785,049, herein incorporated by reference. Also suitable for delivering the dry powders are inhalation devices such as the Turbuhaler™, Rotahaler™, Discus™, Spiros™ inhaler, and the Spinhaler™. Alternatively, the dry powder may be administered via air-assisted devices, such as those that employ the use of a piston to provide air for either entraining powdered medicament, lifting medicament from a carrier screen by passing air through the screen, or mixing air with powder medicament in a mixing chamber with subsequent introduction of the powder to the patient through the mouthpiece of the device. Examples of suitable air-assisted devices are described in U.S. Pat. No. 5,388,572.

The compositions comprising SAP may also be delivered using a pressurized, metered-dose inhaler (MDI) containing a solution or suspension of drug in a pharmaceutically inert liquid propellant, e.g., a chlorofluorocarbon or fluorocarbon. Examples of an MDI include, for example, the Ventolin™ metered-dose inhaler. Suitable propellants, formulations, dispersions, methods, devices and systems are disclosed in U.S. Pat. No. 6,309,623, the disclosure of which is incorporated by reference in its entirety.

In some aspects, an inhalation device comprising SAP is provided. The inhalation device may be any device that is suitable for delivering SAP to the respiratory system of a patient and includes the devices described herein. The inhalation device comprises a pharmaceutical composition of SAP, such as those described herein, and in some embodiments delivers a unit dose of SAP.

In some aspects, the disclosure provides kits, packages and multicontainer units containing SAP pharmaceutical compositions for delivery to the respiratory system. Briefly, these kits include a container comprising SAP and an inhalation device suitable for delivery to the respiratory system. Packaging materials optionally include a label or instructions indicating that the pharmaceutical agent packaged therewith can be used for delivery to the respiratory tract.

### Therapeutic Methods for Delivery of Serum Amyloid P

In one aspect, the disclosure provides methods of administering SAP to the respiratory system of a patient. The term “respiratory system” refers to the anatomical features of a mammal that facilitate gas exchange between the external environment and the blood. The respiratory system can be subdivided into an upper respiratory tract and a lower respiratory tract. The upper respiratory tract includes the nasal passages, pharynx and the larynx, while the trachea, the primary bronchi and lungs are parts of the lower respiratory tract. In some embodiments, methods are provided for the treatment of conditions localized to the respiratory tract, such as the lungs or the nasal cavity.

In one aspect, the disclosure provides methods for treating an SAP-responsive disorder in a patient by administering a therapeutically effective amount of SAP to the respiratory system of a patient in need thereof. In some embodiments, the SAP-responsive disorder is fibrosis. In some embodiments, the SAP-responsive disorder is respiratory fibrosis, i.e., fibrosis of the respiratory tract. The use of SAP as a therapeutic treatment for fibrosis is described in U.S. Patent Application No. 20070243163, which is hereby incorporated by reference.

Fibrosis related disorders that may be amenable to treatment with aerosolized SAP include, but are not limited to, interstitial lung disease, cystic fibrosis, obliterative bronchiolitis, idiopathic pulmonary fibrosis, pulmonary fibrosis from a known etiology, tumor stroma in lung disease, systemic sclerosis affecting the lungs, Hermansky-Pudlak syndrome, coal worker's pneumoconiosis, asbestosis, silicosis, chronic pulmonary hypertension, AIDS-associated pulmonary hypertension, sarcoidosis, chronic asthma, chronic inflammatory airway diseases such as COPD (chronic obstructive pulmonary disease) and emphysema, and acute inflammatory airway diseases such as ARDS (acute respiratory distress syndrome). In some embodiments, aerosolized SAP is administered to treat pulmonary fibrosis.

In some embodiments, the SAP-responsive disorder is a hypersensitivity disorder such as those mediated by Th1 or Th2 responses. In some embodiments, the SAP-responsive disorder is a respiratory hypersensitivity disorder, i.e., a condition related to excessive Th1 or Th2 response affecting the respiratory tract. The use of SAP as a therapeutic treatment for hypersensitivity disorders is also described in U.S. Provisional Application entitled ‘Treatment Methods of Autoimmune Disorders’ by Lynne Anne Murray filed on Mar. 11, 2009, which is hereby incorporated by reference.

Hypersensitivity related disorders that may be amenable to treatment with aerosolized SAP include, but are not limited to, allergen-specific immune responses, allergic rhinitis, allergic sinusitis, allergic asthma, anaphylaxis, food allergies, allergic bronchoconstriction, allergic dyspnea, allergic increase in mucus production in lungs, lung disease cause by acute inflammatory response to allergens (e.g., pollen or a pathogen, such as viral particles, fungi, bacteria), pneumonitis, and chronic obstructive pulmonary disease.

The disclosure provides methods of treating an SAP-responsive disorder comprising administering to a patient a therapeutically effective amount of an aerosolized SAP. The dosage and frequency of treatment can be determined by one skilled in the art and will vary depending on the symptoms, age and body weight of the patient, and the nature and severity of the disorder to be treated or prevented. In certain embodiments, the dosage of SAP will generally be in the range of 0.01 ng to 10 g, 1 ng to 0.1 g, or 100 ng to 10 mg per kg of body weight. In some embodiments, aerosolized SAP is administered to a patient once or twice per day, once or twice per week, once or twice per week, or just prior to or at the onset of symptoms.

Dosages may be readily determined by techniques known to those of skill in the art or as taught herein. Toxicity and therapeutic efficacy of aerosolized SAP may be determined by standard pharmaceutical procedures in experimental animals, e.g., for determining the LD₅₀ and the ED₅₀. The ED₅₀ (Effective Dose 50) is the amount of drug required to produce a specified effect in 50% of an animal population. The LD₅₀ (Lethal Dose 50) is the dose of drug which kills 50% of a sample population. An in vivo model system for studying the effects of intranasal administration of SAP is described in Example 1.

While a patient is being treated with aerosolized SAP, the health of the patient may be monitored by measuring one or more of the relevant indices. Treatment, including dosage and frequency of treatment, may be optimized according to the results of such monitoring. The patient may be periodically reevaluated to determine the extent of improvement by measuring the same parameters, the first such reevaluation typically occurring at the end of four weeks from the onset of therapy, and subsequent reevaluations occurring every four to eight weeks during therapy and then every three months thereafter.

An exemplary index that may be measured during and/or after the course of treatment is pulmonary function. Pulmonary function values and methods to determine said values are well-known in the art. Pulmonary function values include, but are not limited to, FEV (forced expiratory volume), FVC (forced vital capacity), FEF (forced expiratory flow), Vmax (maximum flow), PEFR (peak expiratory flow rate), FRC (functional residual capacity), RV (residual volume), TLC (total lung capacity). FEV measures the volume of air exhaled over a pre-determined period of time by a forced expiration immediately after a full inspiration. FVC measures the total volume of air exhaled immediately after a full inspiration. FEF measures the volume of air exhaled during a FVC divided by the time in seconds. Vmax is the maximum flow measured during FVC. PEFR measures the maximum flow rate during a forced exhale starting from full inspiration. RV is the volume of air remaining in the lungs after a full expiration. In some embodiments, administration of aerosolized SAP increases one or more pulmonary function values.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Intranasal Delivery of SAP

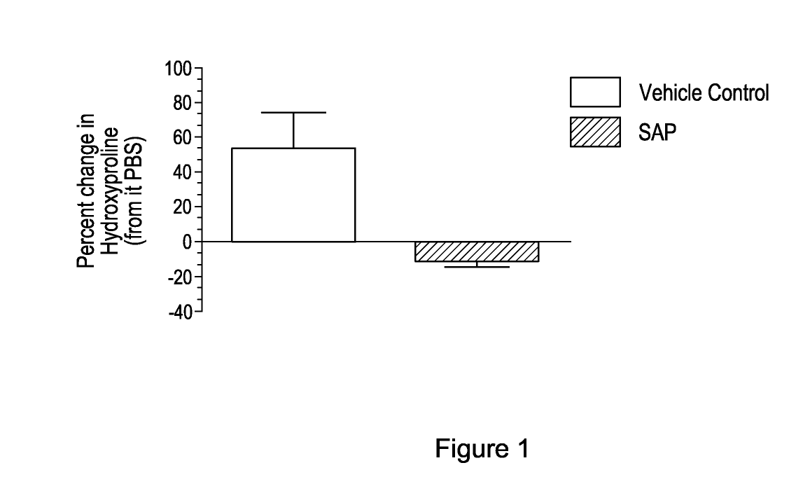

Pulmonary fibrosis was produced in male C57Bl/6 mice. An intratracheal dose (via transoral route) of 0.03 U of bleomycin was administered on Day 0. On study Days 11, 13, 15, 17 and 19 mice in the treated group are dosed intranasally with 8 mg/kg of hSAP (recombinantly produced human SAP) in buffer (10 mM sodium phosphate, 5% sorbitol, pH 7.5). Untreated mice were dosed with buffer. On Day 21 the animals were sacrificed, and total lung collagen was measured using a hydroxyproline assay as described previously (Trujillo et al. Am J Pathol. 2008 172(5):1209-21). Briefly, lung homogenate were incubated with 6 N HCl for 8 hours at 120° C. Following which, citrate/acetate buffer (5% citric acid, 7.2% sodium acetate, 3.4% sodium hydroxide, and 1.2% glacial acetic acid, pH 6.0) and chloramine-T solution (282 mg chloramine-T, 2 ml of n-propanol, 2 ml of distilled water, and 16 ml of citrate/acetate buffer) were added to each digested lung sample. The resulting samples were then incubated at room temperature for 20 minutes before addition of Ehrlich's solution (Aldrich, Milwaukee, Wis.). These samples were incubated for 15 minutes at 65° C., and cooled samples were read at 550 nm in a Beckman DU 640 spectrophotometer. Hydroxyproline concentrations were calculated from a standard curve of hydroxyproline (zero to 100 μg/ml).] Percentage change in total lung collagen was normalized to the amount of collagen in the lungs of mice that had received intratracheal PBS on day 0 (see FIG. 1).

### Example 2

### Detection of hSAP in the Systemic Circulation Following Intranasal hSAP Delivery

C57Bl/6 mice received 100 μl of 20 mg/ml hSAP intranasally and were sacrificed either 6 hours or 24 hours after dosing. A cardiac puncture was performed and resultant plasma analyzed for hSAP levels by ELISA. Lungs were either perfused in situ with PBS via the left ventricle, or not perfused and the lungs then removed en bloc. Tissue was homogenized and hSAP levels measured by ELISA. Table 1 demonstrates the results from the ELISA assays. At both the 6 hour and 24 hour post-intranasal dosing time points, greater levels of hSAP are detected in the lung over plasma.

TABLE 1

| Sample Number | Sample Type | hSAP Levels | Mouse Treatment Description |
|---|---|---|---|
| 08-036, #1 | Mouse Plasma | 0.054 ug/ml | 24 hr post-intranasal dosing |
| 08-036, #2 | Mouse Plasma | BLQ* | 24 hr post-intranasal dosing |
| 08-036, #3 | Mouse Plasma | 0.028 ug/ml | 24 hr post-intranasal dosing |
| 08-036, #7 | Mouse Plasma | BLQ* | 24 hr post-intranasal dosing |
| 08-036, #8 | Mouse Plasma | 0.034 ug/ml | 24 hr post-intranasal dosing |
| 08-036, #9 | Mouse Plasma | 0.039 ug/ml | 24 hr post-intranasal dosing |
| 08-036, #13 | Mouse Plasma | BLQ* | 6 hr post-intranasal dosing |
| 08-036, #14 | Mouse Plasma | 0.113 ug/ml | 6 hr post-intranasal dosing |
| 08-036, #15 | Mouse Plasma | 0.061 ug/ml | 6 hr post-intranasal dosing |
| 08-036, #19 | Mouse Plasma | 0.153 ug/ml | 6 hr post-intranasal dosing |
| 08-036, #20 | Mouse Plasma | 0.113 ug/ml | 6 hr post-intranasal dosing |
| 08-036, #21 | Mouse Plasma | 0.048 ug/ml | 6 hr post-intranasal dosing |
| 08-036, #1 | lung homogenate | 1013 ug/ml | 24 hr, perfused |
| 08-036, #2 | lung homogenate | 0.239 ug/ml | 24 hr, perfused |
| 08-036, #3 | lung homogenate | 16.3 ug/ml | 24 hr, perfused |
| 08-036, #7 | lung homogenate | 15.4 ug/ml | 24 hr, non-perfused |
| 08-036, #8 | lung homogenate | 560 ug/ml | 24 hr, non-perfused |
| 08-036, #9 | lung homogenate | 101 ug/ml | 24 hr, non-perfused |
| 08-036, #13 | lung homogenate | 16.6 ug/ml | 6 hr, perfused |
| 08-036, #14 | lung homogenate | 527 ug/ml | 6 hr, perfused |
| 08-036, #15 | lung homogenate | 157 ug/ml | 6 hr, perfused |
| 08-036, #19 | lung homogenate | 435 ug/ml | 6 hr, non-perfused |
| 08-036, #20 | lung homogenate | 176 ug/ml | 6 hr, non-perfused |
| 08-036, #21 | lung homogenate | 134 ug/ml | 6 hr, non-perfused |

*BLQ, below limit of quantitation

### Example 3

### Aerosolization of hSAP

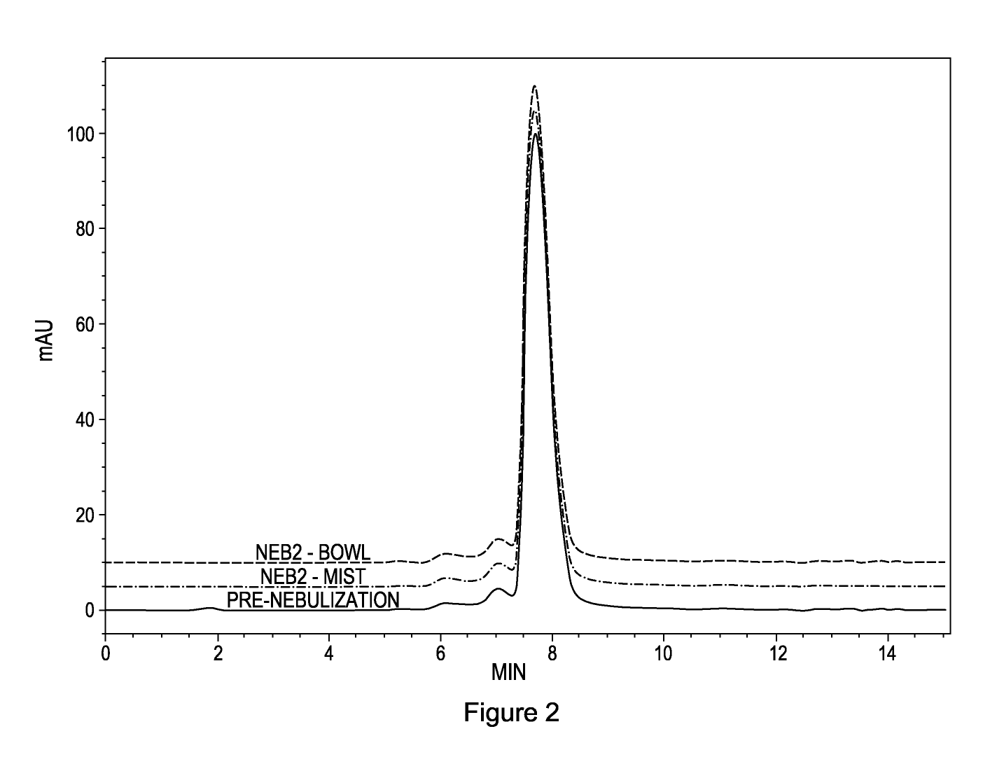

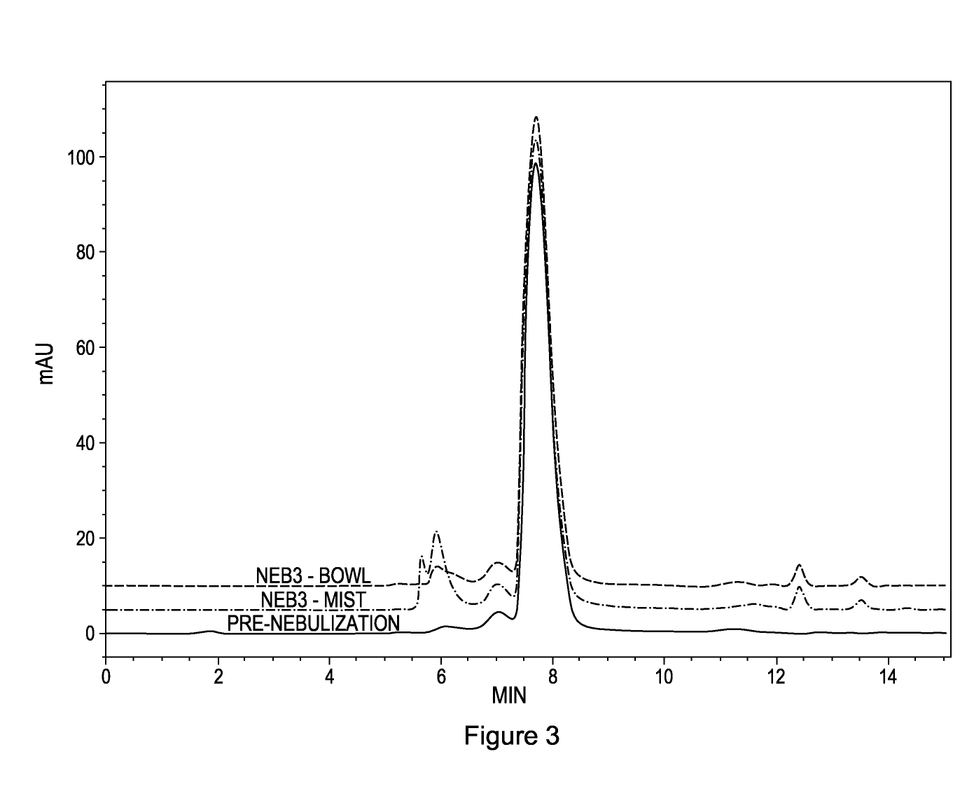

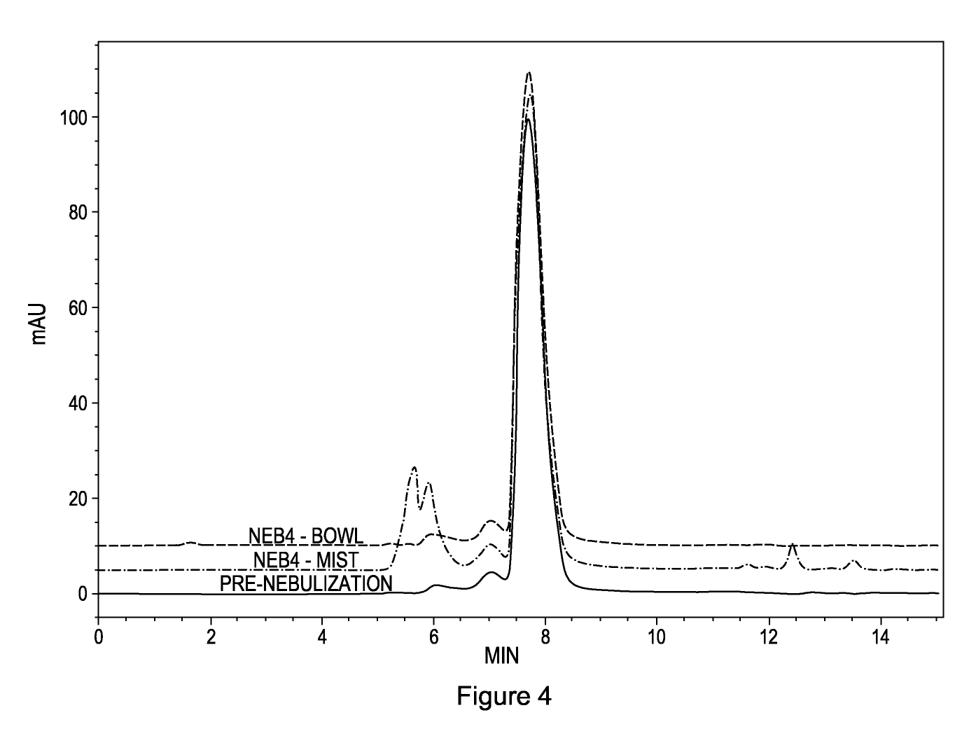

Recombinant human SAP was aerosolized under three different conditions using a DeVilbiss model 3655D nebulizer, see Table 2. Three mL of each sample were introduced into the nebulizer bowl and nebulized for 10 minutes, while the generated aerosol was collected in a 50 mL tube on ice under slight vacuum. Samples of the recovered aerosol (“Recovered”) and of the remainder of the hSAP solution in the nebulizer chamber (“Remainder”) were analyzed by SE-HPLC (size-exclusion high-performance liquid chromatography) and UV absorption to assess the product aggregate content and concentration, respectively. Results are shown in Table 3 and FIGS. 2-4.

TABLE 2

| Sample | mg/ml of hSAP | Buffer |
|---|---|---|
| #1 | 20 | 10 mM sodium phosphate, 5% sorbitol, pH 7.5 |
| #2 | 1 | 10 mM sodium phosphate, 5% sorbitol, pH 7.5 |
| #3 | 1 | 0.9% NaCl |

TABLE 3
UV Concentration Results

| | | Sample | Total | |
|---|---|---|---|---|
| | [hSAP] | Volume | hSAP | Percentage |
| Sample | (mg/mL) | (mL) | (mg) | of feed |
| #1 Recovered | 11.4 | 1.25 | 14 | 22% |
| #1 Remainder | 22.1 | 0.55 | 12 | 19% |
| #2 Recovered | 0.7 | 1.00 | 0.7 | 23% |
| #2 Remainder | 1.1 | 1.20 | 1.3 | 44% |
| #3 Recovered | 0.7 | 1.40 | 1.0 | 32% |
| #3 Remainder | 1.1 | 0.80 | 0.9 | 30% |
| #1 pre-nebulized | 21.7 | 3.0 | 65 | feed |
| #2 pre-nebulized | 1.0 | 3.0 | 3 | feed |
| #3 pre-nebulized | 1.0 | 3.0 | 3 | feed |

On average, 20-30% of the initial hSAP mass was recovered in the aerosol, indicating that it is possible to nebulize hSAP. hSAP concentrations remaining in the nebulizer bowl after 10 minutes did not significantly increase. hSAP nebulized at 1 mg/mL in buffer (10 mM sodium phosphate, 5% sorbitol, pH 7.5) formed significantly more aggregate in the recovered aerosol compared to the 20 mg/mL sample. A second experiment was performed testing a range of hSAP concentrations from 0.5 to 27 mg/mL in 0.9% NaCl with the same nebulizer apparatus and protocol. Product recoveries in the aerosol ranged from 20-28%. In contrast to the first experiment, aggregate content of the recovered aerosol did not show a clear trend with protein concentration.

### Example 4

Chronic allergic airway disease induced by A. fumigatus conidia is characterized by airway hyperreactivity, lung inflammation, eosinophilia, mucus hypersecretion, goblet cell hyperplasia, and subepithelial fibrosis. C57BL/6 mice were similarly sensitized to a commercially available preparation of soluble A. fumigatus antigens as previously described (Hogaboam et al. The American Journal of Pathology. 2000; 156: 723-732). Seven days after the third intranasal challenge, each mouse received 5.0×10⁶ A. fumigatus conidia suspended in 30 μl of PBS tween 80 (0.1%, vol/vol) via intratracheal route.

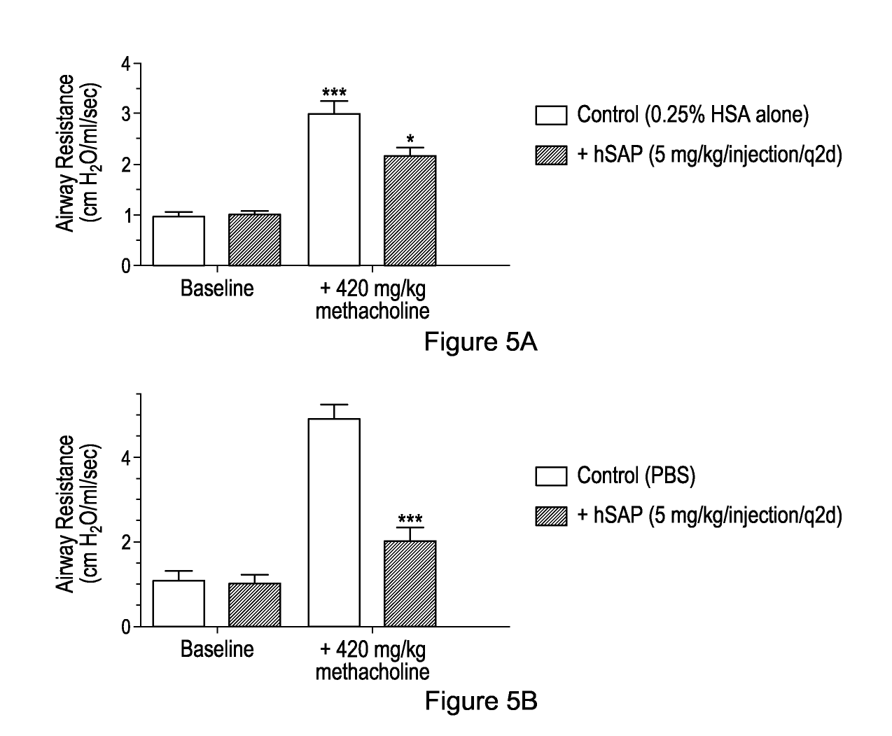

At day 15- and 30-time points (FIGS. 5A and 5B respectively), groups of five mice treated with SAP (5 mg/kg, ip, q2d) or control (PBS, ip, q2d) and analyzed for changes in airway hyperresponsiveness. Bronchial hyperresponsiveness was assessed after an intratracheal A. fumigatus conidia challenge using a Buxco™ plethysmograph (Buxco, Troy, N.Y.). Briefly, sodium pentobarbital (Butler Co., Columbus, Ohio; 0.04 mg/g of mouse body weight) was used to anesthetize mice prior to their intubation and ventilation was carried out with a Harvard pump ventilator (Harvard Apparatus, Reno Nev.). Once baseline airway resistance was established, 420 mg/kg of methacholine was introduced into each mouse via cannulated tail vein, and airway hyperresponsiveness was monitored for approximately 3 minutes. The peak increase in airway resistance was then recorded. At day 15- and 30-time points (FIGS. 5A and 5B respectively), groups of five mice treated with SAP (5 mg/kg, ip, q2d) or control (PBS, ip, q2d) were anesthetized with sodium pentobarbital and analyzed for changes in airway hyperresponsiveness. SAP significantly reduced the amount of AHR in response to intravenous methacholine challenge.

### Example 5

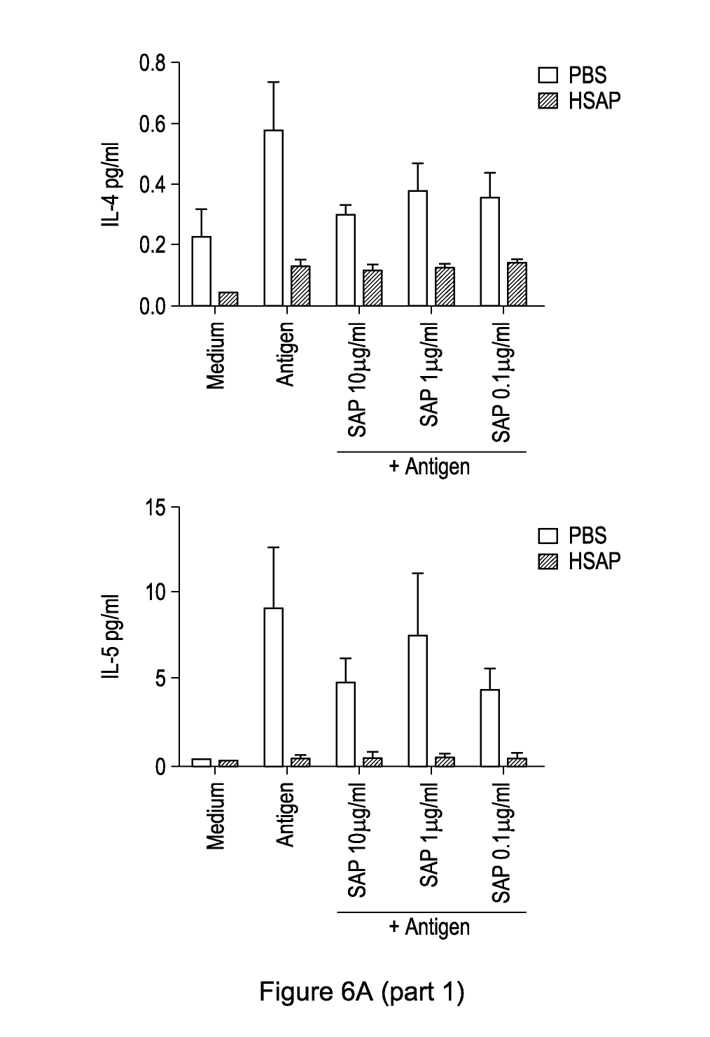

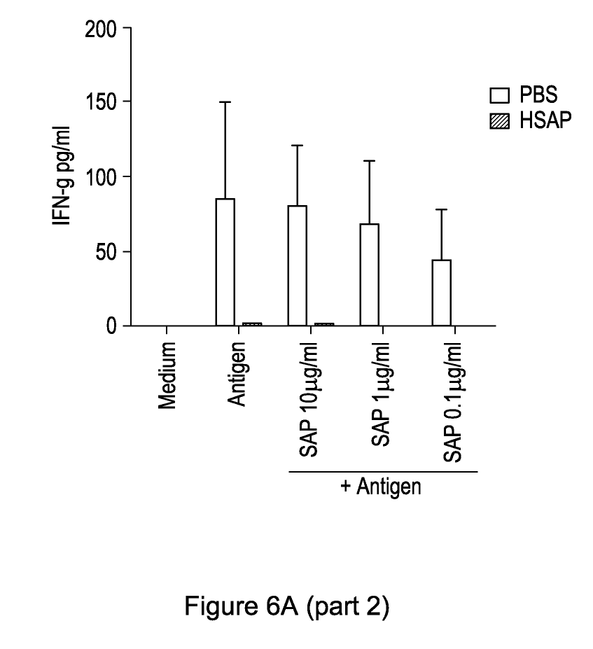

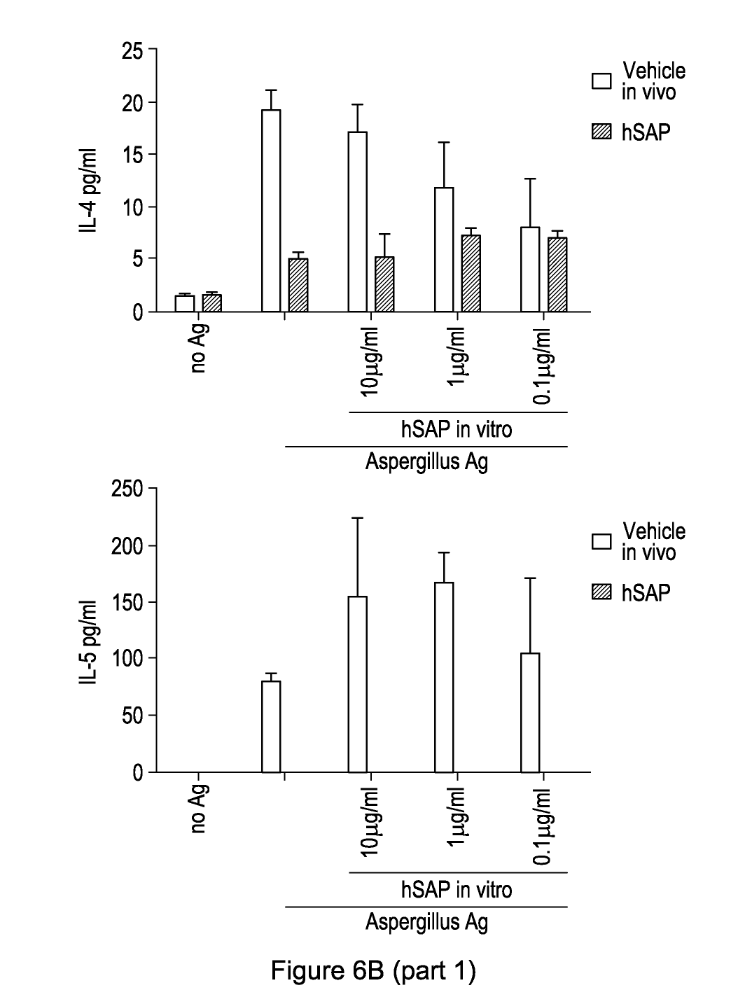

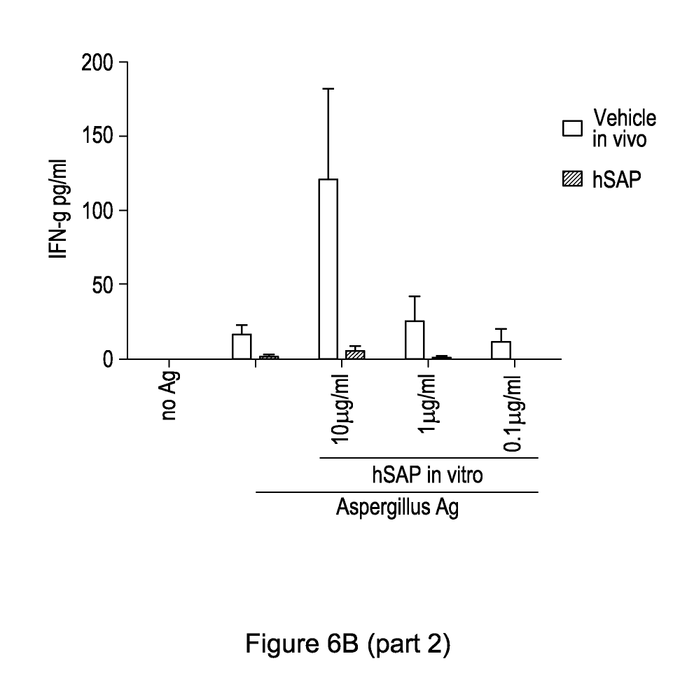

C57BL/6 mice were similarly sensitized to a commercially available preparation of soluble A. fumigatus antigens as above described Animals were treated in vivo with hSAP (8 mg/kg, intranasal (i.n.), 2qd) or control (PBS, in, 2qd) for the last two weeks of the model. At day 15- and 30-time points (FIGS. 6A and 6B respectively), groups of five mice treated were analyzed for changes in cytokine production. Spleen cells were isolated from animals at 15 or 30 days after intratracheal conidia challenge, stimulated with aspergillus antigen, and treated in vitro with hSAP. Splenocyte cultures were quantified (pg/mL) for production of IL-4, IL-5, and INF-γ.

### Example 6

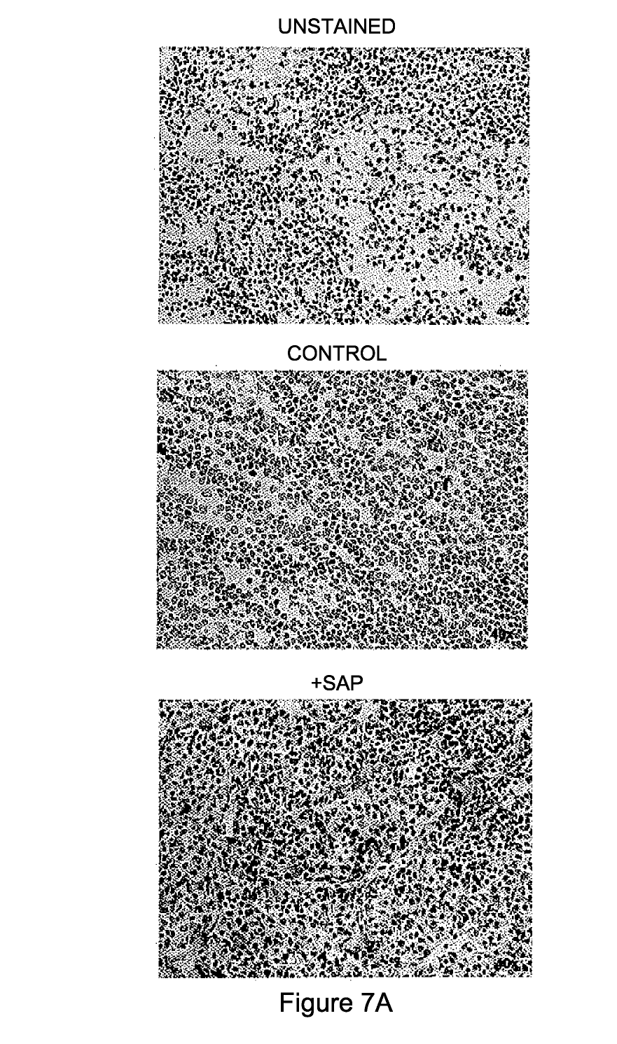

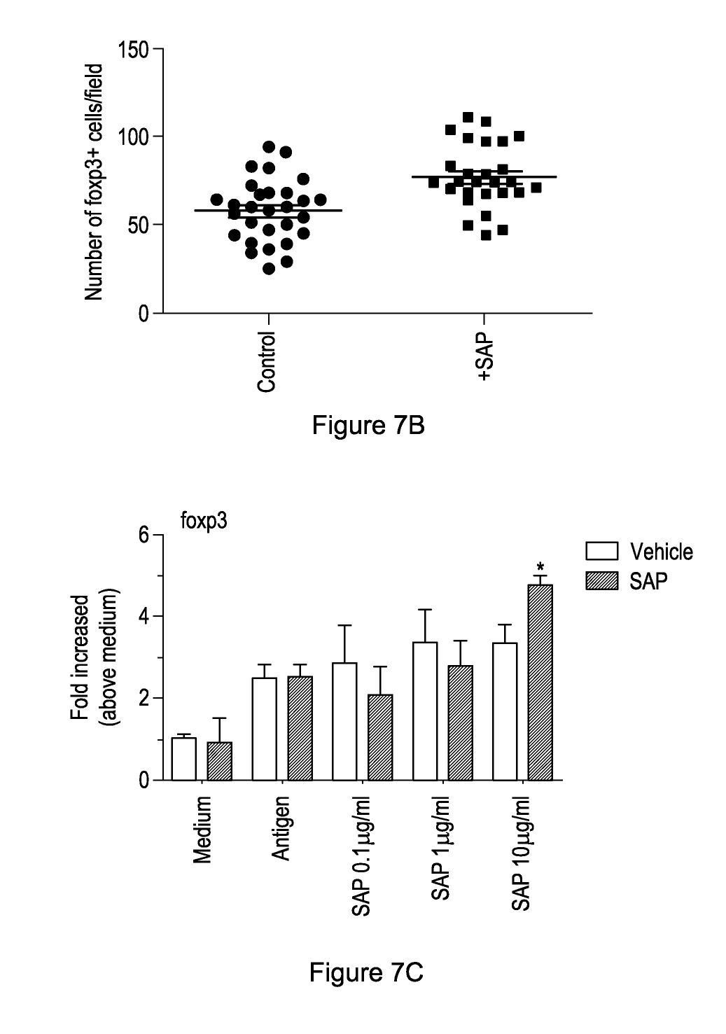

C57BL/6 mice were similarly sensitized to a commercially available preparation of soluble A. fumigatus antigens as above described. At day 15, the amount of FoxP3 expression was determined in pulmonary draining lymph nodes or splenocyte cultures. Pulmonary lymph nodes were dissected from each mouse and snap frozen in liquid N₂ or fixed in 10% formalin for histological analysis. Histological samples from animals treated with SAP (8 mg/kg, i.n., 2qd) or control (PBS, in, 2qd) were stained for FoxP3 (FIG. 7A), and the number of FoxP3+ cells were quantified relative to each field examined (FIG. 7B). Purified splenocyte cultures were stimulated with Aspergillus antigen in vitro in the presence or absence of SAP in vitro (0.1-10 μg/ml) for 24 hours. Total FoxP3 expression was quantitated using real time RT-PCR (FIG. 7C).

### Example 7

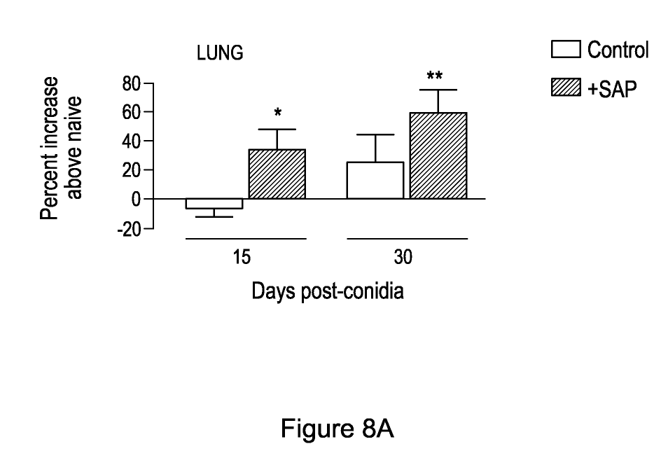

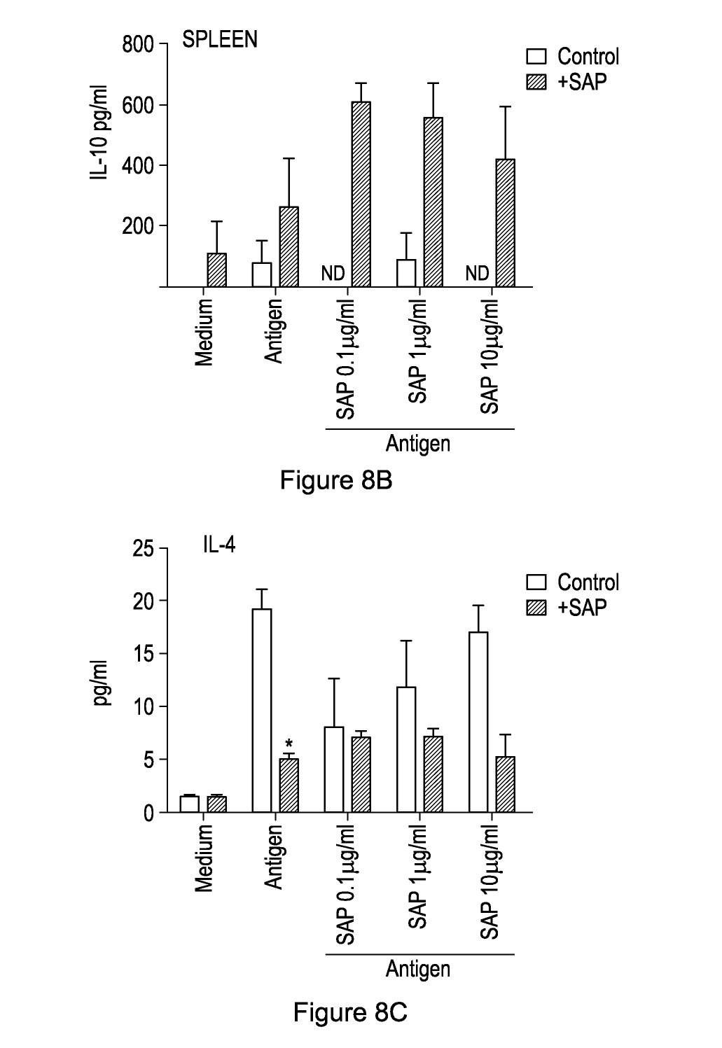

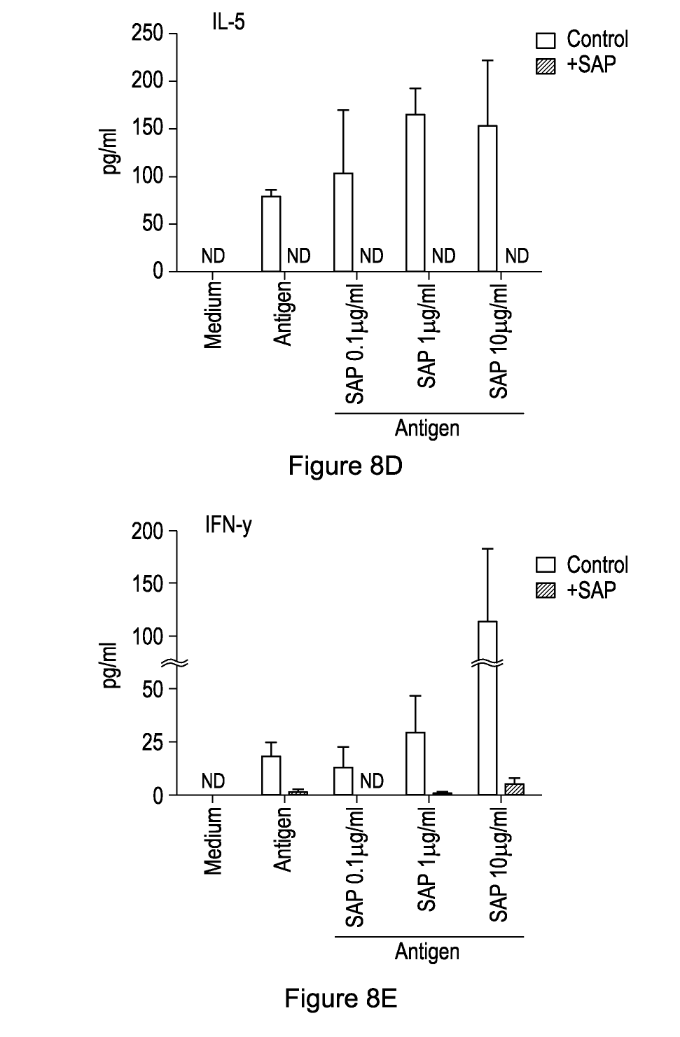

The effects of SAP in vivo and in vitro on IL-10 and antigen recall were examined, see FIG. 8. Mice were sensitized and challenged with Aspergillus fumigatus in vivo and treated with control (PBS, ip, q2d open bars) or SAP (5 mg/kg, ip, q2d, filled bars) on days 15-30 post-live conidia challenge. At day 30, mice were sacrificed. A) Total lung IL-10 was measured by luminex. B-E) Single cell splenocyte cultures were stimulated in vitro with Aspergillus fumigatus antigen, in the presence or absence of SAP (FIG. 8). Cell-free supernatants were assessed for B) IL-10, C) IL-4, D) IL-5 and E) IFN-γ protein levels by ELISA. The data demonstrates that SAP treated animals (ip, q2d on days 15-30) had enhanced levels of IL-10 in the lungs in comparison to asthma control (PBS, ip, q2d, on days 15-30) and levels were comparable to that in naive, non-allergic lung (FIG. 8). Splenocytes from SAP treated mice have a reduced Th1 or Th2 antigen recall response and increased IL-10. As there is also an increase in FoxP3 expression, this data indicates that SAP induces regulatory T cells within the setting of allergic airways disease.

We claim:

1. A microparticulate system for delivery of serum amyloid P (SAP) to the respiratory system comprising biodegradable microparticles comprising SAP and a pharmaceutically acceptable carrier.

2. An aerosol comprising serum amyloid P (SAP).

3. The aerosol of claim 2, further comprising a lipid.

4. The aerosol of claim 2, wherein the aerosol is liquid and comprises from about 0.5 mg/ml to about 100 mg/ml of SAP.

5. The aerosol of claim 2, further comprising from about 0.1% to about 2% NaCl.

6. The aerosol of claim 2, further comprising from about 1 mM to about 20 mM sodium phosphate.

7. The aerosol of claim 2, comprising dry particles that comprise from about 10% to about 100% w/w of SAP.

8. The aerosol of claim 7, wherein the aerosolized particles have a mass median aerodynamic diameter of less than about 10 microns.

9. A dry powder pharmaceutical composition suitable for delivery to the respiratory system, comprising serum amyloid P (SAP) and a pharmaceutically acceptable carrier.

10. The dry powder pharmaceutical composition of claim 9, wherein particles of the powder have a mass median aerodynamic diameter of less than about 10 microns.

11. A method of administering serum amyloid P (SAP) to a patient in need thereof, comprising aerosolizing a pharmaceutical composition comprising SAP for inhalation into the respiratory system of the patient.

12. A method of treating respiratory fibrosis in a patient, comprising administering to a patient in need thereof a therapeutically effective amount of an aerosolized serum amyloid P (SAP) pharmaceutical composition.

13. A method of treating a respiratory hypersensitivity disorder in a patient, comprising administering to a patient in need thereof a therapeutically effective amount of an aerosolized serum amyloid P (SAP) pharmaceutical composition.

14. The method of claim 11, wherein the pharmaceutical composition comprises biodegradable microparticles comprising SAP.

15. The method of claim 11, wherein the pharmaceutical composition is a dry powder suitable for respiratory delivery.

16. The method of claim 11, wherein the pharmaceutical composition is administered with a dry powder inhaler.

17. An inhalation device comprising a pharmaceutical composition comprising serum amyloid P (SAP).

18. The inhalation device of claim 17, wherein the pharmaceutical composition comprises biodegradable microparticles comprising SAP.

19. The inhalation device of claim 17, wherein the pharmaceutical composition is a dry powder suitable for respiratory delivery.

20. The inhalation device of claim 19, wherein the dry powder comprises from about 10% to about 100% w/w of SAP.

21. The inhalation device of claim 17, wherein the inhalation device is selected from a metered-dose inhaler; a dry powder inhaler, a nasal delivery device; or a jet, ultrasonic, pressurized or vibrating porous plate nebulizer.
